(19) United States
(12) Patent Application Publication
(10) Pub. No.: US 2002/0086313 A1
(43) Pub. Date: Jul. 4, 2002

(54) Application of bioinformatics for direct study of unculturable microorganisms

(76) Inventor: John J. Kilbane II, Woodstock, IL (US)

Correspondence Address: Mark E. Fejer, Gas Technology Institute, 1700 South Mount Prospect Road, Des Plaines, IL, 60018, US

(21) Appl. No.: 09/960,698

(22) Filed: Sep. 21, 2001

Related U.S. Application Data
(60) Provisional application No. 60/235,095, filed on Sep. 25, 2000.

(51) Int. Cl. C12Q 1/68; G06F 19/00; C12P 19/34

(52) U.S. Cl. 435/6; 435/91.2; 702/20

(57) ABSTRACT

A method for identifying unculturable microorganisms in which at least one bacterial cell from an environmental sample containing a plurality of microorganisms is isolated, at least one DNA fragment from the at least one bacterial cell is amplified, cloned into at least one E. coli vector and sequenced, resulting in identification of at least one DNA sequence. The at least one DNA sequence is compared with existing DNA databases, resulting in identification of the at least one DNA sequence as derived from either an unculturable microorganism or a known microorganism.

### CROSS-REFERENCE TO RELATED APPLICATION

This application claims the benefit of provisional U.S. patent application Ser. No. 60/235,095, filed Sep. 25, 2000.

### BACKGROUND OF THE INVENTION

1. Field of the Invention

This invention relates to the application of bioinformatics to enable the direct study of unculturable microorganisms. More particularly, this invention relates to a method for identifying unculturable microorganisms so as to enable study of such unculturable microorganisms in their natural environment, which allows for a better appreciation of the contributions of these microorganisms to soil ecology and provides the potential for growing such microorganisms in the laboratory. The method of this invention is applicable to the study of all unculturable microorganisms. As used herein, the term “unculturable microorganism(s)” refers to microorganisms that are currently incapable of being grown as pure cultures under laboratory conditions.

2. Description of Related Art

It is estimated that 99% or more of all microorganisms are currently classified as unculturable. The primary problem is a current lack of techniques for the study of unculturable microorganisms. The existence of unculturable microorganisms is known primarily from DNA-DNA reassociation studies and PCR-based studies employing total DNA extracted from soil and other environmental samples and the amplification of the highly conserved prokaryotic 16S and 5S rRNA gene sequences. DNA complexity can be estimated from a kinetic analysis of the reassociation of total DNA extracted from environmental samples. Such studies indicate that there are approximately 4000 to 13,000 different bacterial species in a gram of average soil. The majority of microbial diversity is located in that part of the community which cannot be isolated and cultured by standard techniques. Cultivation and cultivation-independent techniques combined can rarely account for more than 100 total species in an individual soil sample. This is all the more remarkable considering that there is seldom any overlap between the lists of species identified by cultivation and cultivation-independent techniques. DNA-DNA reassociation experiments provide a means of estimating the total number of bacterial species present in a sample, but to obtain information about individual bacterial species, an examination of 16S rRNA gene sequences or other highly conserved sequences is most often performed and current techniques only allow the gathering of very limited data from only a small fraction of species actually present in an environmental sample. Clearly the ability to access and characterize biodiversity in environmental samples using existing techniques is limited and needs improvement.

The amplification of rRNA sequences from DNA mixtures derived from environmental samples introduces unintended biases to the results obtained. The abundance and physiological state of different bacterial species varies considerably as do the efficiencies of cell lysis. Additionally, bacterial species differ in the number of rrn operons within their genome, and there are different template efficiencies relative to the primers used. An additional problem with the amplification of nucleic acids obtained from environmental samples is the interference from humic acids and other substances that may significantly decrease the efficiencies of these procedures. A further complication with obtaining rRNA gene sequences from DNA mixtures using the PCR is the formation of chimeric molecules that are artifacts and not representative of any living species. However, the main limitation in obtaining biodiversity data from DNA mixtures using PCR techniques is that DNA molecules present in the greatest abundance will be preferentially amplified. Thus, it is doubtful if bacterial species present in low abundance will be represented in the relatively few rDNA clones that will be sequenced from amplicons derived from mixed culture DNA. It is apparent that in order to obtain information about the vast majority of microorganisms that constitute microbial diversity, techniques must be employed that permit the detection and examination of all of the bacteria that may be present in a sample and not just the 1% that can be grown in the laboratory using current technology or the few percent that may comprise the most abundant species in a given environmental sample that can be detected by current cultivation-independent techniques.

Some studies of unculturable microorganisms involve the extraction of DNA from environmental samples and the cloning of DNA fragments into vectors so that they can be maintained and studied in E. coli or other laboratory friendly microorganisms. Typically, DNA fragments of 30 to 100 kb can be cloned. These DNA fragments can be sequenced and compared to known DNA sequence databases to identify DNA fragments derived from unculturable microorganisms and to locate protein coding sequences within these fragments. In some cases, the expression of genes from unculturable microorganisms has been accomplished. Frequently, promoters from one species fail to function in other species, but the expression of some genes from their native promoters has been observed in E. coli. It is also possible to clone individual genes derived from unculturable microorganisms and place them in expression vectors for E. coli or other hosts. However, these known experimental approaches only provide indirect and incomplete information regarding unculturable microorganisms. The size of a typical bacterial chromosome is about 4000 kb, so a DNA fragment of 40 kb represents only 1% of the genetic information in a bacterial cell. It is, thus, apparent that there is a need for direct and complete information about unculturable microorganisms.

As previously suggested, the practical benefits that may be realized from the direct study of unculturable microorganisms are substantial as it will result in an increased knowledge base for the entire field of microbiology. An example is antibiotics. The majority of antibiotics used in the treatment of infectious diseases of humans and animals are derived from a variety of known microorganisms. However, the clinical effectiveness of most antibiotics has declined in recent years due to the development of resistance in disease causing microorganisms. This problem has been addressed by the isolation of new classes of antibiotics through the study of previously obscure/unknown microorganisms and by producing chemical derivatives of known antibiotics. However, the rate of discovery of new antibiotics is declining as culturable microorganisms have been thoroughly examined. Similarly, many antibiotics currently in use are already third and fourth generation chemical derivatives of antibiotic molecules originally isolated from microorganisms. It will, thus, be apparent that the ability to produce clinically effective new antibiotics through the chemical modification of existing antibiotics is nearly exhausted.

Gaining information about unculturable microorganisms will provide scientists with the ability to clone and express increased amounts of genes from unculturable microorganisms in laboratory-friendly bacterial hosts and/or to grow increasing numbers of unculturable microorganisms in the laboratory. These novel microorganisms, which vastly outnumber the species of currently known microorganisms, will undoubtedly contain multiple novel antibiotics which can then be expected to be widely used in the treatment and prevention of infectious diseases. Likewise, the availability of previously unculturable microorganisms will provide improvements and new capabilities in environmental remediation, agriculture, biotechnology, chemistry and other industries.

### SUMMARY OF THE INVENTION

It is, thus, one object of this invention to provide a method for identifying unculturable microorganisms.

It is another object of this invention to provide a method by which unculturable microorganisms can be studied directly.

It is another object of this invention to provide a method by which increased amounts of DNA sequence data from the genomes of unculturable microorganisms can be obtained.

It is yet another object of this invention to provide a method for identifying unculturable microorganisms which permits the detection and examination of all of the microorganisms in a sample.

These and other objects of this invention are addressed by a method for identifying unculturable microorganisms comprising the steps of isolating at least one bacterial cell from an environmental sample comprising a plurality of microorganisms, amplifying at least one DNA fragment from the at least one bacterial cell, cloning the at least one DNA fragment into at least one E. coli vector, sequencing the at least one DNA fragment, resulting in identification of at least one DNA sequence, and comparing the at least one DNA sequence with existing DNA databases, resulting in identification of the at least one DNA sequence as either an unculturable microorganism or a known microorganism.

In accordance with one preferred embodiment of this invention, short oligonucleotides are used as “universal” PCR primers that target multiple genetic loci that will enable amplification of the DNA fragments from most, if not all, unculturable microorganisms. With sufficient DNA sequence information derived from the genomes of individual species of unculturable microorganisms, bioinformatics is used to design species-specific DNA probes suitable for directly studying the unculturable microorganisms in their natural environment. Data obtained regarding the genetics, and particularly the nutritional requirements and physiology, of individual species of previously unculturable microorganisms will enable new culturing techniques to be developed so that at least some previously unculturable microorganisms can be grown in the laboratory.

### BRIEF DESCRIPTION OF THE DRAWINGS

These and other objects and features of this invention will be better understood from the following detailed description taken in conjunction with the drawings wherein:

FIG. 1 is a schematic illustration showing the use of various fluorescent dyes to achieve fractionation of a mixed microbial population obtained from an environmental sample;

FIG. 2 is a diagram showing flow cytometry data of the same cell population derived from the saturated zone of a hydrocarbon-contaminated site before staining with the fluorescent dye Fluorescein DHPE; and

FIG. 3 is a diagram showing flow cytometry data of the same cell population derived from the saturated zone of a hydrocarbon-contaminated site after staining with the fluorescent dye Fluorescein DHPE.

### DETAILED DESCRIPTION OF THE PRESENTLY PREFERRED EMBODIMENTS

The objective of this invention is the study of individual species of unculturable microorganisms in their natural environment. Until now, the study of unculturable microorganisms has been limited to methodologies that provide only indirect and incomplete information. Until now, there has been no comprehensive study of individual species of unculturable microorganisms in their natural environment. Until now, techniques that can be used to directly study unculturable microorganisms, including species-specific DNA probes have been largely unknown, particularly as regards the majority of genetic loci in the majority of unculturable microorganisms.

When total DNA from an environmental sample is used to clone fragments, some of these DNA fragments will be from unculturable microorganisms. However, there is no way of knowing if any of the many fragments thus obtained are derived from the same or different species. Therefore, the largest amount of DNA that can be obtained from a single species of unculturable microorganism is limited to the size of the largest single DNA fragment that can be obtained, which is generally about 30 to 100 kb. Moreover, all of the DNA sequence information about an unculturable microorganism comes from a single region of the chromosome. Using the method of this invention, multiple DNA fragments of various lengths are derived from multiple loci throughout the chromosome of an individual species of unculturable microorganism. These DNA sequences comprise hundreds, if not thousands, of kb of DNA sequence data that provide a much more thorough sampling of the genome of the unculturable microorganism species, which, in turn, allows multiple species-specific DNA probes to be designed targeting many genes in that species. Then, using species-specific DNA probes for a wide assortment of genes, a more accurate picture of the physiological response of unculturable microorganisms to various environmental factors can be determined.

The prior art describes the cloning of random DNA fragments from unculturable microorganisms, and in some cases attempts are made to express genes derived from unculturable microorganisms in E. coli or other well-studied bacteria. However, the expression of genes from foreign species is not usually successful using the native promoter of that gene. Even if expression of a gene from an unculturable microorganism is achieved in E. coli or some other laboratory strain of bacteria, the physiological effect/relevance of that gene product to the original bacterial host will most often be unknown and not amenable to study in E. coli. Most gene products/proteins have intricate interactions with other components in the cell and are most often subject to complex interdependent regulation mechanisms that can only be studied directly in the species where the gene originates (or some highly related species). This invention allows genes to be studied directly in their natural host so that achieving expression of the gene will not be an issue and the intricacies of gene regulation and cell physiology can be studied in a way that would likely never be possible in E. coli or other well studied bacterial species.

Until now it has been thought by those skilled in the art that the direct study of unculturable microorganisms is not possible because of the very low amount of DNA or RNA that can be obtained from an individual species present in a complex mixture of DNA and RNA of all species present in an environmental example. It is also thought by those skilled in the art that there is no way to sort out which DNA and/or RNA fragments present in such a mixture originate from individual species of microorganisms because of the lack of species-specific probes. Moreover, without multiple species-specific probes derived from many genes from the same species, it is thought that only sparse and generally indirect information about the physiology of unculturable microorganisms can be obtained.

The method of this invention comprises five key steps: 1) isolation of individual bacterial cells from environmental samples; 2) use of short oligonucleotides as “universal” PCR primers targeting multiple genetic loci that enable amplification of DNA from unculturable microorganisms; 3) cloning of the resulting DNA fragments into E. coli vectors and sequencing of the DNA of each fragment to obtain DNA sequence data derived from individual cells of unculturable microorganisms; 4) with sufficient DNA sequence information derived from the genomes of individual species of unculturable microorganisms, using bioinformatics to design species-specific DNA probes suitable for direct study of unculturable microorganisms in their natural environment; and 5) applying data obtained from the use of species-specific DNA probes regarding the physiology of individual species of previously unculturable microorganisms to develop culturing techniques so that at least some previously unculturable microorganisms can be grown in a laboratory.

In accordance with one embodiment of this invention, individual bacterial cells are isolated from environmental samples, such as soil, using a micromanipulator (obtainable from Narishige in Tokyo, Japan) or a flow cytometer (obtainable from Becton-Dickinson, Mountainview, Calif.) equipped with a cell sorting device. Because 99% or more of all bacteria are unculturable microorganisms, the direct isolation of individual bacterial cells from environmental samples is an appropriate means for obtaining unculturable microorganisms. Individual bacterial cells thus obtained are then subjected to amplification by PCR using one or more short oligonucleotides of arbitrary sequence as “universal” primers. An appropriate length for the oligonucleotides used as universal primers is in the range of about 8 to about 20 base pairs (bp). Short individual oligonucleotides can be used to prime the PCR rather than the pair of primers required in conventional PCR. Alternatively, pairs of oligonucleotides can be employed to increase the likelihood of amplifying a greater percentage of given bacterial genomes. To further increase the efficiency of amplifying given bacterial genomes, high-GC content primers, high-AT content primers and/or one high-GC content primer and one high-AT content primer are preferred to obtain unique DNA fragments from all unculturable microorganisms.

The DNA fragments thus obtained are cloned into appropriate E. coli vectors to facilitate subsequent analysis. The DNA fragments thus obtained all derive from the chromosome of a single microorganism and, thus, from a single species of microorganism. The DNA sequences of each cloned DNA fragment are then determined. Because individual bacterial cells are studied, several hundreds or even thousands of kb of unique DNA fragments may be obtained from several loci from individual species of unculturable microorganisms. The DNA sequences are then compared with existing DNA sequence databases to confirm that they originate from unculturable microorganisms and to identify DNA sequences that can serve as species-specific DNA probes. The species-specific DNA regions may then be used to design PCR primer pairs targeting each unique DNA sequence and to prepare hybridization probes/DNA chip arrays. In accordance with one embodiment of this invention, the preferred size for the species-specific DNA primers for PCR and RTPCR experiments is in the range of about 20 to about 50 bp. The size of species-specific gene probes for use in hybridization experiments and the fabrication of DNA microarrays/gene chips is in the range of about 20 to about 2000 bp.

Environmental samples, such as soil, may then be subjected to various conditions such as the addition of various carbon and nitrogen sources, alteration of pH, aerobic and anaerobic conditions, addition of environmental pollutants and the like. Total DNA and/or RNA samples may then be obtained from these treated environmental samples and the species-specific primers and DNA probes used in PCR, RT-PCR and microarray hybridization/gene expression experiments to obtain data concerning the response of unculturable microorganisms to various environmental changes. In this way, the unculturable microorganisms can be studied directly in their natural environment and data thus obtained may be used as the basis for determining the requirements for growth of at least some of these microorganisms under laboratory conditions as axenic cultures.

Even if growth of a microorganism under laboratory conditions is not achieved, a large percentage of the genome of such organisms will be available for study as cloned fragments in E. coli.

### EXAMPLE

An environmental sample derived from the saturated zone of a hydrocarbon-contaminated site was processed to obtain a cell suspension which was then subjected to flow cytometry/cell sorting after staining with the lipid-staining dye fluorescein DHPE to yield two populations of cells: low fluorescence and high fluorescence. It was found that about 12 to 14% of the total cell population was stained with this dye, but to varying degrees. The gating parameters of the cell sorting device were adjusted to stringent conditions to allow only the most intensely stained cells in the mixture, which comprised about 1% of the total cell population, to be separated as a discreet sub-population of bacterial cells. This mixture of cells subsequently was further sorted to isolate individual bacterial cells, which were then placed in individual test tubes/wells. The cells were lysed to release chromosomal DNA which was then subjected to PCR using a 10-mer oligonucleotide as a primer. The DNA fragments thus amplified were then cloned into E. coli vectors and the DNA sequence of each DNA fragment determined. These DNA sequences were then compared with the DNA sequences of all characterized microorganisms to determine if these DNA sequences, in fact, originate from previously unculturable microorganisms and to define specific DNA regions/sequences that can be used as species-specific probes for each species of unculturable microorganism studied. These species-specific DNA sequences were then used in hybridization experiments to analyze the effects of various environmental parameters on the growth and activity of individual species of unculturable microorganisms.

In addition to culturability, another significant problem encountered in investigations of microbial ecology is the relative abundance of various bacterial species. If a bacterial species can be grown as a pure culture under laboratory conditions, then it is relatively straightforward to determine the ability of that culture to contribute to the remediation of a contaminant and to determine the effects of environmental parameters. However, as previously indicated, if a culture cannot be grown in the laboratory, then it is difficult to conveniently and reliably obtain information about the potential of a culture for remediation of contaminants or its response to environmental parameters. The use of dilution culture techniques as a means of increasing the number of cultures that can be grown in the lab has been demonstrated. Likewise, the use of novel media can allow an increased number of microorganisms to be grown in the laboratory. Nevertheless, it is clear that the ability to grow a greater percentage of microbial species in the laboratory can be improved. While it may be impractical to attempt to identify novel media that can be used to grow each species of yet-to-be-cultivated microorganisms, the systematic use of dilution culture techniques is capable of providing substantial rewards in the growth of many yet-to-be-cultivated bacterial species.

The phenomenon underlying the success of dilution culture techniques to allow the growth of novel microbial communities/microorganisms is known as competitive exclusion. The relative abundance of a bacterial species in a microbial community is not only based on its ability to utilize the nutrients present and tolerate the prevailing environmental conditions, but also on the ability of a species to compete with all the other members of the microbial community. When a mixed bacterial culture is diluted and then used to inoculate a growth experiment, those microbial species that were originally present in low abundance may no longer be present, as a result of which the composition for limiting nutrients is changed. As a result, it has been possible to isolate some bacterial species from dilution culture experiments that were not isolated from the same inoculum in undiluted form.

It is unclear what fraction of microbial species can be grown in the laboratory using currently available techniques, but it is certainly a larger number than has been grown thus far. However, and importantly, no systematic effort to overcome the effects of competitive exclusion and determine the maximum number of novel/yet-to-be-cultivated species that can be grown from environmental samples has been performed.

The importance of the phenomenon of competitive exclusion is perhaps best illustrated by the wide-spread observation that when the microbial community in environmental samples is assessed by culture-based techniques as well as by cultivation-independent techniques, most often there is little or no overlap in the list of microbial species identified in the same sample using the two approaches. Cultivation-independent techniques generally employ DNA purified from the mixed microbial community and then examine 16S r-RNA sequences either by directly cloning DNA fragments or, more commonly, by PCR amplification. The 16S rRNA genes that are detected are derived exclusively from the most abundant bacterial species present in the original sample.

The fact that cultivation-based techniques result in the isolation of a completely different subset of bacterial species demonstrates that current laboratory cultivation procedures do not faithfully mimic real-world environmental conditions so that the most abundant species present in environmental samples are victims of competitive exclusion in laboratory microbial growth experiments. Those species that do grow under laboratory conditions are often those that had relatively low abundance in the original sample. Therefore, if environmental samples are diluted, low abundance species are lost from the mix, thereby eliminating some of the source of competitive exclusion in laboratory growth experiments, resulting in the isolation of novel bacterial species.

A primary limitation of the dilution culture approach is that the low abundance species will always be lost, yet low abundance species comprise the majority of biodiversity. An alternative means of altering the composition of mixed microbial populations is to fractionate the population based on various physiological parameters using flow cytometry. Flow cytometry, and particularly fluorescence-activated cell sorting (FASC), is capable of precisely sorting mixed microbial populations that differ in some regard. There are a variety of fluorescent dyes that can be used to selectively stain protein, lipids, DNA, and even AT-rich or GC-rich DNA (as well as other target molecules). This invention employs differential staining of mixed bacterial populations combined with FASC to obtain sub-populations of mixed bacterial cultures that can be used to avoid competitive exclusion in bacterial growth experiments and to provide DNA samples that are enriched for the presence of rare microbial species.

Bacterial sub-populations produced by flow cytometry, especially when employing dyes that do not effect cell viability, could be extremely fertile sources for the investigation of microbial ecology and biodiversity. Bacterial species will respond differently to dyes targeting protein, lipids or other cellular components and when different dyes are used, flow cytometry can yield bacterial sub-populations that differ significantly from the original mixed culture, from each other, and from anything that can be produced by the dilution culture. Importantly, some of these sub-populations will be enriched for bacterial species that were present in low abundance in the original sample. If these sub-populations of bacterial cultures are subsequently subjected to cultivation and cultivation-independent means of investigation, it is highly likely that novel bacterial species will be grown in the laboratory and analyses of 16S r-RNA molecules will reveal to a greater extent the biodiversity present in the original sample. If a bacterial culture is first fractionated by flow cytometry and then subjected to dilution culture experiments, it is possible that additional novel bacterial species will be successfully grown in the laboratory.

Flow cytometry can be used to obtain sub-populations of bacteria that differ in composition from that of original environmental samples. These bacterial sub-populations should alter the dynamics of inter-species competition and competitive exclusion in microbial growth experiments, thereby allowing a wider array of microbial species to be grown in the laboratory. Additionally, these bacterial sub-populations can also be analyzed by cultivation-independent techniques affording a more detailed view of the biodiversity in environmental samples. In this way, both culturable and unculturable microorganisms can be studied directly in their natural environment and data can be obtained that may lead to an improved understanding of the biodiversity of environmental samples of all kinds.

Environmental samples are analyzed as is and after being divided into various sub-populations based on physiological parameters using flow cytometry and fluorescence-activated cell sorting. The species composition of these microbial sub-populations are then investigated by cultivation and cultivation-independent methods. The cultivation of novel bacterial species is aided by the use of dilution culture and the use of numerous media with compositions intended to mimic the natural environment from which the samples are derived. Molecular analyses determine the most abundant species present in each bacterial sub-population by cloning 16S rRNA genes and determining their DNA sequences. Additionally, hybridization experiments are performed to determine the overall level of biodiversity present in each bacterial sub-population.

Filtration is used to concentrate bacteria from environmental samples and then various staining procedures are used to selectively stain protein, lipids, viable cells, DNA, AT-rich DNA, and GC-rich DNA. Flow cytometry is used to sort bacterial cells into various sub-populations. Through the use of various fluorescent dyes and fluorescence activated cell sorting, it should be possible to process environmental samples to obtain large collections of bacterial cells that possess various staining properties. Even though in many environmental samples a few bacterial species can predominate, comprising from 20% to 90% of the cells, the combination of various dyes with fluorescence activated cell sorting should result in cell collections/libraries that are diverse and contain high percentages of unculturable microorganisms.

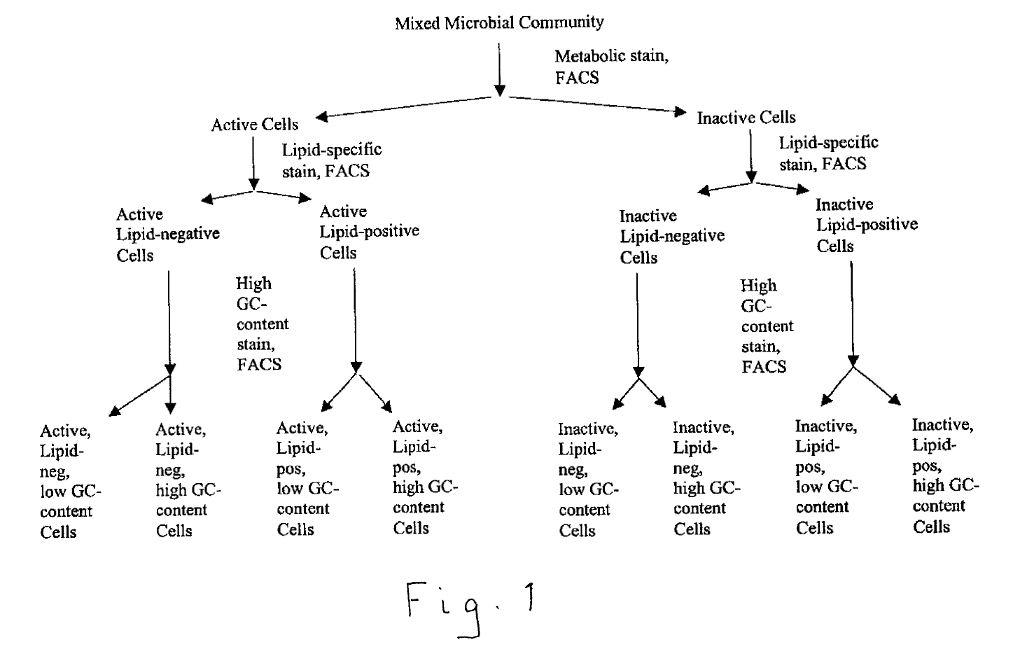

An objective of this invention is to demonstrate that fluorescence-activated cell sorting (FACS) can be used to fractionate mixed microbial populations in ways that facilitate the analysis of biodiversity. There are numerous fluorescent dyes available that selectively bind to various biological molecules and produce fluorescent signals that can be readily detected by flow cytometry, thereby allowing the separation of fluorescing from non-fluorescing cells. A schematic illustration of the use of various fluorescent dyes to achieve fractionation of a mixed microbial population obtained from an environmental sample is shown in FIG. 1. Many cells present in environmental samples may not be metabolically active and the uptake of certain dyes can be used to selectively label metabolically inactive bacterial cells. A specific example of a dye that can be used to selectively stain viable cells is DiBAC4 (Catalogue number B-438, Molecular Probes, Eugene, Oreg.). Dye-treated populations of cells are then processed by FACS to allow active cells to be separated from inactive cells. A possible next step might be to use a different fluorescent dye that selectively binds to lipids and has a fluorescent signal at a different wavelength than the dye previously used. When this lipid-specific fluorescent dye is subsequently used to stain the active and the inactive microbial cell populations each cell mixture can again be processed by FACS resulting in four sub-populations of bacterial cells as shown in FIG. 1. Specific examples of lipid staining dyes that can be used for the purpose of selectively staining bacterial lipids are BODIPY FL C16 (catalogue number D-3821, Molecular Probes, Eugene, Oreg.), 16-(9-anthroyloxy) palmitic acid (catalogue number A-39, Molecular Probes, Eugene, Oreg.), or fluorescein DHPE (catalogue number F362, Molecular Probes, Eugene, Oreg.). Similarly yet another dye that fluoresces at a unique wavelength relative to the dyes previously used can be used to again stain each of the cell sub-populations. In the example shown in FIG. 1, a dye that selectively binds to GC-rich DNA is used and, after processing by FACS, eight sub-populations of bacterial cells are obtained. Specific examples of fluorescent dyes that can be used to stain nucleic acids in bacteria are Hoechst 33342 (catalogue number H-3570, Molecular Probes, Eugene, Oreg.) and SYBR Green (catalogue number S-7563, Molecular Probes, Eugene, Oreg.).

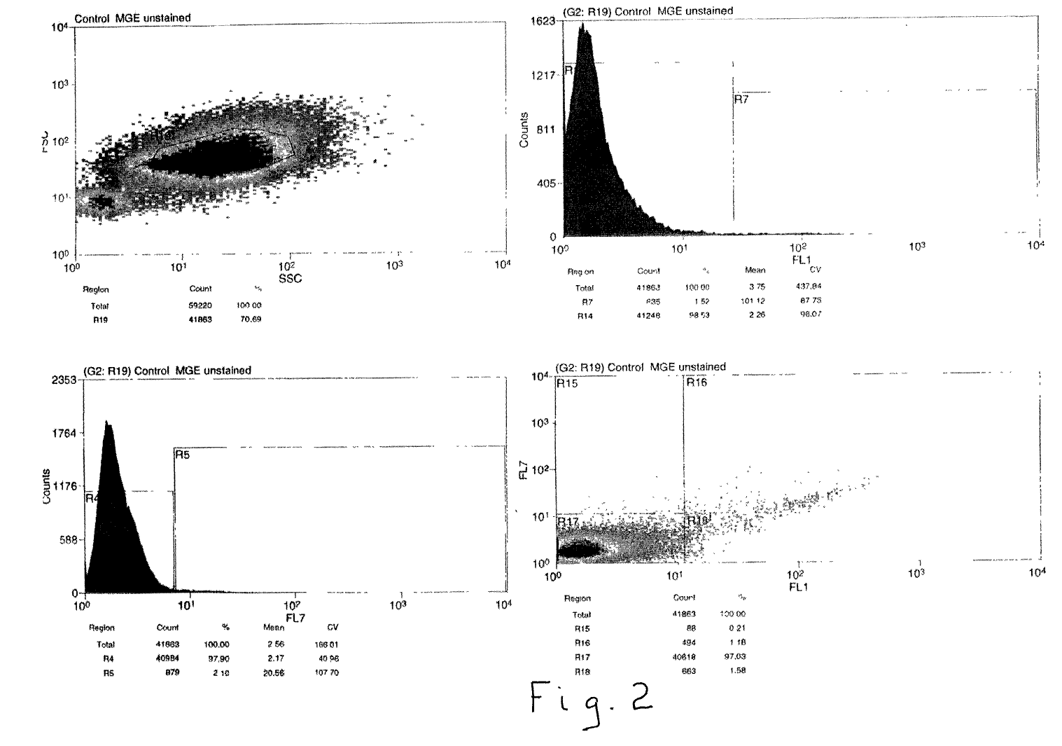

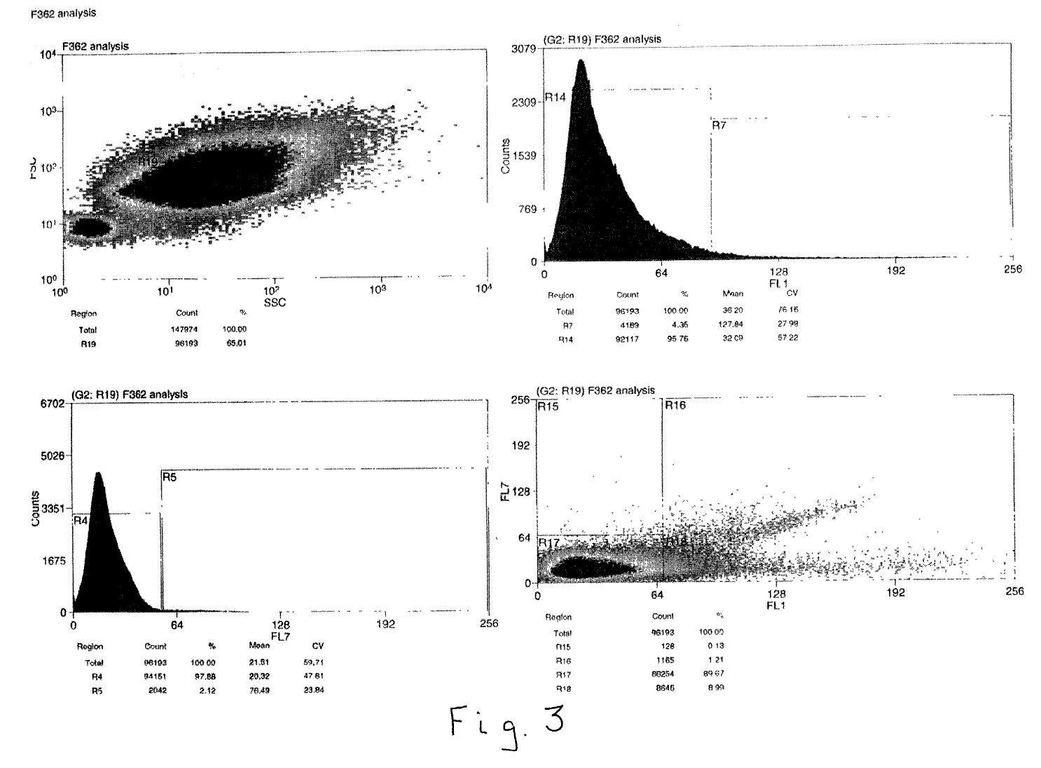

FIGS. 2 and 3 show flow cytometry data of the same cell population derived from the saturated zone of a hydrocarbon-contaminated site before and after staining with the fluorescent dye Fluorescein DHPE (catalogue number F-362, Molecular Probes, Eugene, Oreg.) that targets lipids. A comparison of the two sets of graphs demonstrates that the use of the lipid-specific dye enables preferential staining of a portion of the population so that it has different optical properties as compared with the original unstained sample. These differences in optical properties allow fluorescence-activated cell sorting to be used to obtain a subset of the original population that comprises a portion of the original population that could not have been obtained in any previously known way. The best demonstration of the differences can be appreciated by looking at the graphs at the lower right of the figures that are labeled (G2:R19) for Control MGE unstained and F362 analysis respectively. These graphs plot FL 1 versus FL7, which are two fluorescence channels that are appropriate for the analysis of cells stained with Fluorescein DHPE which fluoresces at 519 nanometers maximum emission. In particular these graphs are divided into four quadrants with quadrant R18 being of greatest interest. The quantity of cells in the original unstained sample that were in the R18 quadrant were 1.58% of the total population, whereas after staining with F362 dye, 8.99% of the population ends up in the R18 quadrant and the overall appearance of the two graphs is different. By adjusting the gating parameters of the cell sorting device, only those cells in the far right hand portion of quadrant R18 in the F632-stained population are isolated and recovered cells corresponding to about 1% of the cell population that could not be detected or isolated uniquely in the unstained sample. The unstained sample is estimated to have about 10,000 different species of bacteria present, but genetic analysis will typically only allow the detection of a few (20 to 100) species that are present in the greatest abundance. The sorted cells obtained after FACS of the F362-stained cells yielded a sub-population that is about 1% of the original sample and therefore may contain about 100 different bacterial species, but most of these 100 species are expected to be those that would have been lost in the crowd in the original sample.

The cell fractionation scheme illustrated in FIG. 1 is just an example. In practice the goals of cell fractionation are two-fold: to obtain sub-populations of cells that are substantially free from those bacterial species that were most abundant in the original sample, and to obtain sub-populations of cells that are substantially free of those bacterial species that are most readily cultivated from the original sample. If the most abundant bacterial species can be removed, then the remaining bacterial population will be substantially enriched for rare (low abundance) bacterial species. Such a sub-population of bacterial cells may subsequently allow novel microorganisms to be cultured under laboratory conditions and they most certainly make good sources from which to prepare 16S rRNA libraries and genomic libraries. Similarly, if those bacterial species that are most readily cultivated from the original sample are removed, then the probable sources of competitive exclusion will also be removed, making it more likely that novel bacterial species can subsequently be cultivated under laboratory conditions. These sub-populations are also good sources from which to prepare genomic libraries.

Some fluorescent dyes have been shown to have little or no effect on the viability of prokaryotic cells while other dyes require permeabilization of cells and will not allow the recovery of viable cells after FACS. The initial steps in using FACS to fractionate microbial populations focus on those dyes that will permit the subsequent use of cell sub-populations in microbial growth experiments for the potential isolation of species that have not previously been cultivated. Microbial populations and sub-populations may also be subjected to FACS using dyes/procedures that do not allow cells to remain viable, and in these instances the resulting microbial sub-populations may be subjected to molecular analyses to create libraries of 16S rRNA genes to characterize the biodiversity present in these samples and to generate genomic libraries from which a multitude of biotechnology products may be derived.

To prepare microbial sub-populations in which the most abundant bacterial species present in the original sample are selectively removed, fluorescent-labeled DNA probes targeting DNA sequences unique to each abundant species can be used to selectively label these abundant cells followed by FACS. Suitable species-specific probes can be prepared from the variable regions of the 16S RNA genes and from other species-specific probes targeting other genes. This is the most convenient approach as the required DNA sequence data will be available. An alternative approach for creating species-specific probes with even greater specificity that target the most abundant bacterial species is to create genomic libraries from total DNA extracted from microbial populations and perform colony hybridization to detect clones containing DNA fragments that include 16S rRNA genes. These DNA fragments will be derived predominantly from the most abundant bacterial species which can be confirmed by sequencing these fragments. These DNA fragments will contain genes in addition to the 16S rRNA gene and species-specific probes can be prepared targeting unique chromosomal genes and/or intergene spacer regions.

In another application of this invention, after species-specific probes for unculturable microorganisms have been developed, fluorescence activated cell sorting can be used to demonstrate the use of flow cytometry to isolate additional cells of specific species of unculturable microorganisms. For these studies, environmental samples are first permeabilized to permit the entry of fluorescent DNA or peptide nucleic acid probes. It may be advantageous to use peptide nucleic acid probes rather than DNA probes due to their better ability to permeate cells, resistance to nucleases and proteases, and higher binding affinities.

DNA sequence data derived from presumptive unculturable microorganisms will initially be compared with genomic sequences/databases of known microorganisms to determine if the sequence of the 16S rRNA gene confirms the identity of a given cell as an unculturable microorganism and to determine the relatedness to known microbial species. Subsequent analysis of DNA sequence data derived from unculturable microorganisms will focus on identifying unique regions of genes/open reading frames that enable species-specific probes to be designed that can be used in FACS experiments to obtain additional samples of particular species of unculturable microorganisms and for use in PCR and/or hybridization/microarray experiments to characterize unculturable microorganisms.

It will be of great interest and utility to obtain additional cells of a given species of unculturable microorganisms to demonstrate that this technique can facilitate the further analysis of specific species of unculturable microorganisms. It will always be useful to obtain additional DNA sequence data, and the ability to obtain a relatively abundant and pure sample of the bacterial species of interest will enable a variety of genetic and biochemical tests to be performed. Unfortunately, the use of species-specific DNA probes requires the permeabilization of bacterial cells so that viable cells cannot be obtained for study. However, it is possible that the analysis of the cell surface of individual species of unculturable microorganisms may lead to a future fluorescence activated cell sorting method to isolate viable cells of that species. If a sufficient quantity of cells of a given species of unculturable microorganism are obtained by the use of species-specific probes and fluorescence activated cell sorting, then it may be possible to develop antibodies that will allow the subsequent purification of viable cells of that species. This is yet another example of how this invention can be used to detect and characterize biodiversity of microorganisms.

While in the foregoing specification this invention has been described in relation to certain preferred embodiments thereof, and many details have been set forth for the purpose of illustration, it will be apparent to those skilled in the art that the invention is susceptible to additional embodiments and that certain of the details described herein can be varied considerably without departing from the basic principles of this invention.

I claim:

1. A method for identifying unculturable microorganisms comprising the steps of:
isolating at least one bacterial cell from an environmental sample comprising a plurality of microorganisms;
amplifying at least one DNA fragment from said at least one bacterial cell;
cloning said at least one DNA fragment into at least one E. coli vector;
sequencing said at least one DNA fragment, resulting in identification of at least one DNA sequence; and
comparing said at least one DNA sequence with existing DNA databases, resulting in identification of said at least one DNA sequence as one of an unculturable microorganism and a known microorganism.

2. A method in accordance with claim 1, wherein said at least one DNA fragment is amplified by a polymeric chain reaction (PCR) using at least one universal primer.

3. A method in accordance with claim 2, wherein said universal primer is an oligonucleotide of arbitrary sequence.

4. A method in accordance with claim 3, wherein said oligonucleotide comprises in a range of about 8 bp to about 20 bp.

5. A method in accordance with claim 2, wherein said at least one universal primer is one of a high-GC content primer and a high-AT content primer.

6. A method in accordance with claim 2, wherein a pair of said at least one universal primer comprises two primers selected from the group consisting of high-GC content primers, high-AT content primers and mixtures thereof.

7. A method in accordance with claim 2, wherein said at least one universal primer comprises a random mixture of oligonucleotides having a common length and differing in DNA sequence.

8. A method in accordance with claim 1 further comprising identifying at least one said DNA sequence suitable for use as a species-specific DNA sequence.

9. A method in accordance with claim 1, wherein said at least one bacterial cell is isolated from said environmental sample with a micromanipulator.

10. A method in accordance with claim 1, wherein said at least one bacterial cell is isolated from said environmental sample using flow cytometry.

11. A method in accordance with claim 8, wherein at least one hybridization probe/DNA chip array is prepared using said species-specific DNA probe.

12. A method in accordance with claim 8, wherein at least one PCR primer pair suitable for targeting at least one unique said DNA sequence is prepared using said species-specific DNA sequence.

13. A method in accordance with claim 11, wherein said species-specific DNA probe comprises in a range of about 20 bp to about 2000 bp.

14. A method in accordance with claim 12, wherein said PCR primers used to amplify said species-specific DNA sequence comprises in a range of about 20 bp to about 50 bp.

15. A method in accordance with claim 1, wherein a plurality of said DNA fragments of various lengths are derived from multiple loci throughout a chromosome of said unculturable microorganism.

16. A method in accordance with claim 6, wherein additional said environmental samples are subjected to at least one condition, at least one of total DNA and/or total RNA is obtained from said additional said environmental samples, and said species-specific DNA probe is used in methods selected from the group consisting of PCR, RT-PCR and microarray hybridization/gene expression, resulting in generation of data concerning responses of said unculturable microorganisms to said at least one condition.

17. A method in accordance with claim 1, wherein at least one fluorescent dye is used to differentially stained said plurality of microorganisms which are subsequently processed by flow cytometry and cell sorting to produce at least two sub-populations that differ in terms of at least one of species composition and species relative abundance from said environmental sample.

18. A method in accordance with claim 17, wherein at least one of said sub-populations is subjected to dilution culture experiments utilizing a plurality of bacterial growth media, resulting in growth of at least one species of previously unculturable microorganism.

19. A method in accordance with claim 17, wherein at least one of said sub-populations is subjected to genetic analysis to detect and analyze 16S rRNA sequences to obtain improved data regarding the biodiversity of said environmental sample.

20. A method in accordance with claim 17, wherein at least one of said sub-populations is used to prepare at least one genomic library.

21. A method in accordance with claim 17, wherein at least one of said sub-populations is further processed by FACS to obtain at least one individual bacterial cell.
